(19) United States
(12) Patent Application Publication
(10) Pub. No.: US 2018/0156769 A1
(43) Pub. Date: Jun. 7, 2018

(54) SYSTEMS AND METHODS FOR MONITORING AND MANAGING SOLUTION SAMPLES

(71) Applicant: AQUASEND, Vista, CA (US)

(72) Inventor: Kristin Elliott, Vista, CA (US)

(73) Assignee: AQUASEND, Vista, CA (US)

(21) Appl. No.: 15/803,703

(22) Filed: Nov. 3, 2017

Related U.S. Application Data
(60) Provisional application No. 62/431,381, filed on Dec. 7, 2016.

(51) Int. Cl. G01N 33/18

(52) U.S. Cl. CPC G01N 33/1886

(57) ABSTRACT

Disclosed are various embodiments of a monitoring apparatus and a method for obtaining solution sample measurements. The monitoring apparatus may include a hollow body and a top cover to seal the hollow body to provide a waterproof monitoring apparatus. Other embodiments may also include a weighted bottom portion beneath the hollow body and a sensor exposed to the environment to monitor and acquire data from a solution sample.

### RELATED APPLICATIONS

This application claims priority to U.S. Provisional Patent Application No. 62/431,351 filed on Dec. 7, 2016, the contents of which is incorporated herein by reference in its entirety.

### TECHNICAL FIELD

The disclosed technology relates generally to monitoring and managing data acquired from solution samples. More specifically, the present disclosure is directed towards monitoring and managing the acquired data from solution samples collected in real time for water quality sampling.

### BACKGROUND

Measuring and analyzing the contents and quality of solution samples is important, especially when the solution sample is from an aquaculture environment. Aquaculture refers to the breeding, rearing and harvesting of plants and animals in all types of water environments, including tanks, ponds, rivers, lakes and oceans.

Because the aquaculture is an environment that houses living organisms, researchers and farmers must constantly analyze the water sample to ensure that the temperature, oxygen content, pH and the like are well within the ranges suitable for a healthy and thriving environment.

As such, researchers and famers alike need real-time monitoring and analysis systems to provide such monitoring data and results, especially considering that even the slightest environment changes can stress and even debilitate living organisms housed in a tank or aquaculture environment. Thus, real-time monitoring and analysis systems can be seen as essential tools to help increase the health and production of the contained living organisms. Additionally, real-time water quality monitoring may even help save electricity and other overhead costs, as researchers and farmers may be able to control and eliminate the need for the constant use of aerators and bubblers, and instead, only utilize them when needed.

However, current technology does not provide efficient or cost-effective real time aquaculture monitoring and analysis solutions. Furthermore, most technologies require the use of extensive wiring so that the monitoring systems are physically integrated to a land based control used.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an illustration of an monitoring apparatus that monitors solutions in real time according to certain embodiments in the provided disclosure.

FIG. 2 is an illustration of an monitoring apparatus that monitors solutions in real time according to certain embodiments in the provided disclosure.

FIG. 3 is an illustration of a bottom view of a monitoring apparatus according to certain embodiments in the provided disclosure.

FIG. 4 is an illustration of a computer control system that implements the monitoring apparatus according to certain aspects of the provided disclosure.

FIG. 5 is a cross-sectional view of an monitoring apparatus that monitors solution samples in real time according to certain embodiments in the provided disclosure.

FIG. 6 is an illustration of a sensor and a sensor wiper of a monitoring apparatus according to certain embodiments of the provided disclosure.

FIG. 7 is a flow chart of an exemplary process for monitoring a body of water according to certain embodiments of the provided disclosure.

FIG. 8 is a diagram depicting an example computing module used to implement features according to certain embodiments of the provided disclosure.

The figures are not intended to be exhaustive or to limit the disclosed technology to the precise form disclosed. It should be understood that the disclosed technology can be practiced with modification and alteration, and that the disclosed technology be limited only by the claims and the equivalents thereof.

### DETAILED DESCRIPTION OF THE EMBODIMENTS

The following description is non-limiting and is made merely for the purpose of describing the general principles of the disclosed embodiments. Numerous specific details are set forth to provide a full understanding of various aspects of the subject disclosure. It will be apparent, however, to one ordinarily skilled in the art that various aspects of the subject disclosure may be practiced without some of these specific details. In other instances, well-known structures and techniques have not been shown in detail to avoid unnecessarily obscuring the subject disclosure.

The disclosure provides an apparatus and method for monitoring a body of water, aquaculture environment, solvent or solution sample. From herein, the sample may be sourced from what will generally referred to as a “body of water,” which may include, but is not limited to, any of a body of water, aquaculture environment, solvent or solution to describe any solution being collected and measured by the disclosed apparatus.

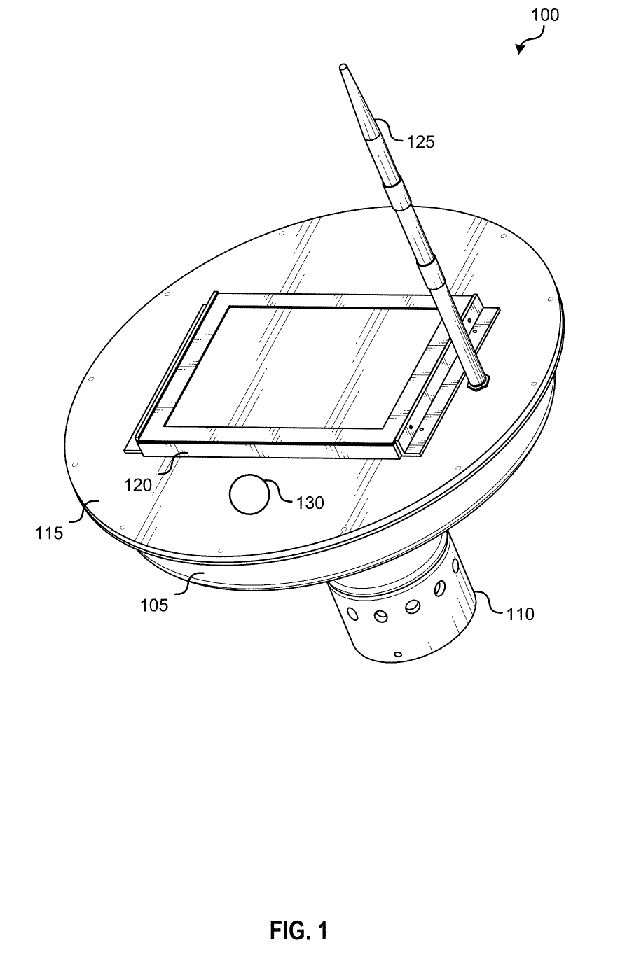

FIG. 1 is an illustration of an monitoring apparatus 100 that monitors solutions in real time according to certain embodiments in the provided disclosure. By way of example only, the monitoring apparatus 100 may be a buoy so that it floats when placed in a body of water. In some embodiments, the monitoring apparatus 100 may be configured to have a weighted bottom 110 section and a wide top surface area so that the monitoring apparatus 100 is stable and will not tip over when afloat.

The monitoring apparatus 100 may be configured to have a hollow body 105, which may allow the monitoring apparatus 100 to be lightweight so that it can be carried and transported with ease. By way of example, the monitoring apparatus 100 may range anywhere from 5 to 15 pounds, which is significantly lighter than most other current monitoring apparatuses.

Additionally, the monitoring apparatus 100 may be configured to provide a waterproof housing so that all electronic hardware and circuits may be securely sealed within the inside compartment of the monitoring apparatus 100. By way of example, the monitoring apparatus 100 may have a hollow body 105, where the electronic hardware, circuits, sensors, and the like may be placed. Furthermore, the monitoring apparatus 100 may include a hard top 115 that is securely placed over the hollow body 105. The hard top 115 may be tightly sealed so that the monitoring apparatus 110 is waterproof.

By way of example, the hard top 115 of the monitoring apparatus 100 may float above the water line while the weighted bottom section 110 is to be placed beneath the water level. The weighted bottom 110 may be evenly weighted to ensure that the monitoring apparatus 100 is balanced. In some embodiments, the hard top 115 may have a solar panel 120. The solar panel 120 may charge an internal battery (not shown here) placed within the hollow body 105 of the monitoring apparatus 100. The internal battery may be used to power the electric components, such as the sensors, motors, and any other electrical components within the monitoring apparatus 100 that need to be powered.

Furthermore, the hard top 115 may also include an antenna 125, which may allow the monitoring apparatus 100 to be in connected to WiFi and other communicational networks, such as radio, cellular, etc. As a result, any data collected from the sensors in the monitoring apparatus 100 may be transmitted to an offsite data storage server or computer system. In other instances, the antenna 125 may be used to send signals to other monitoring apparatuses 100, such as when attempting to sync them or communicate with the other monitoring apparatuses 100. In other instances, the antenna 125 may send signals to other devices, such as those used to help regulate the specific environmental conditions of the solution. By way of example, such devices may include an aerator, thermostat, cooler, heater, bubbler, and any other device used to regulate conditions of the body of water. Thus, the devices may automatically shut down, turn-off, or be adjusted based on the data and signals provided by the monitoring apparatus 100. More detail is provided below.

Thus, the monitoring apparatus 100 may be configured to operate wire free such that there are no wires connecting the monitoring apparatus 100 to a land based control device or system. In other words, the disclosed monitoring apparatus 100 may be free floating and integrated with a cloud computing model to manage and operate the monitoring apparatus 100.

In some embodiments, the monitoring apparatus 100 may include a status indicator 130, which may display lights of various different colors. Depending on the light color, this may visually indicate important information to the user or personnel. For example, in the instance that the status indicator 130 is red, this may indicate that the body of water is currently in less than ideal conditions as determined by the user's set determined parameters. Thus, the red light may provide visual indication to the user that the environment of the contained body of water needs immediate attention. Such scenarios, by way of example, may be when the monitoring apparatus 100 detects low or high levels of oxygens and temperatures above or below a pre-determined threshold. In another example, in the instance that the status indicator 130 is green, this may indicate that the body of water is currently being maintained in ideal conditions as determined by the user's set determined parameters. In another example, in the instance that the status indicator 130 is yellow, this may indicate that the body of water is nearing borderline conditions that are close to less than ideal conditions as determined by the user's set determined parameters.

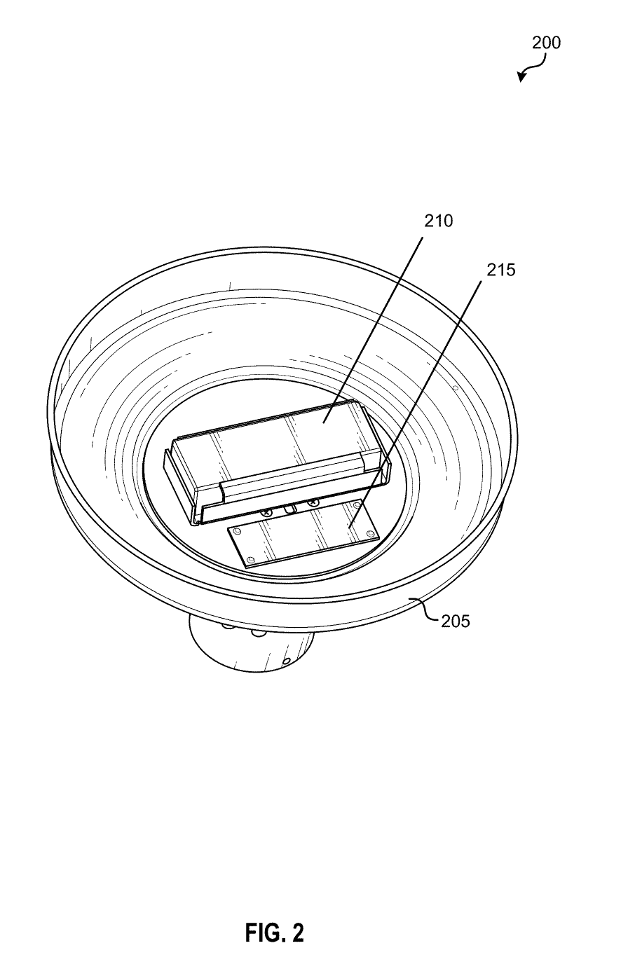

FIG. 2 is an illustration of the hollow body 205 of the monitoring apparatus 200. The hollow body 205 may be configured to float on a body of water in a stable manner and decrease the likelihood of the monitoring apparatus 200 flipping over.

The hollow body 205 may include an internal rechargeable battery 210 and a communication board 215. The internal rechargeable battery 210 may be connected to a solar power panel (not shown here) as illustrated in at least FIG. 1. Additionally, the communication board 215 may be configured to perform high level and data repository operations in communication with a web server software. The communication board 215 may decode and encode communications to and from a data storage server accessible via a web browser. Thus, the communication board 215 may ensure that the data collected from the monitoring apparatus 200 is stored on a data storage server and in communication with a web browser.

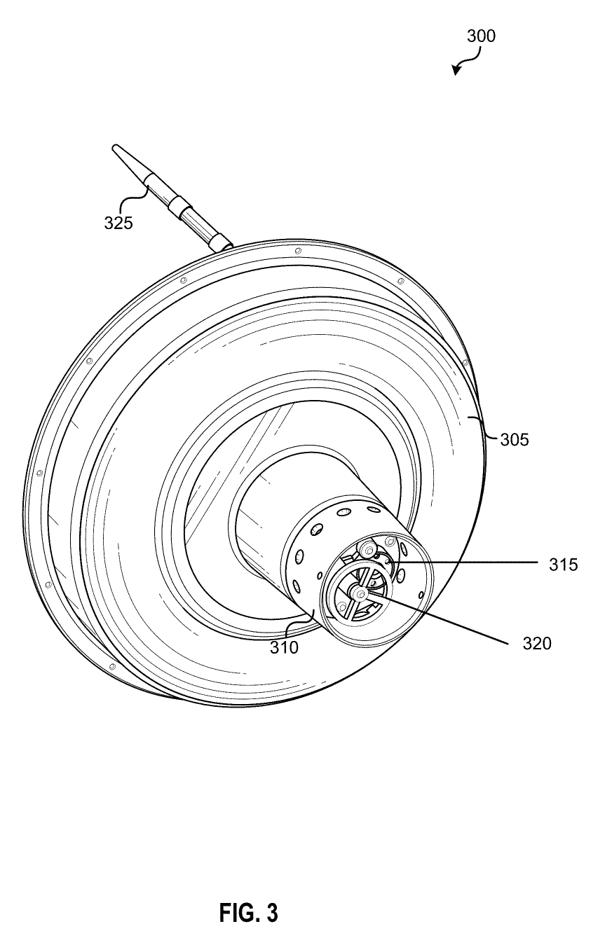

FIG. 3 is an illustration of an monitoring apparatus 300 that monitors solution samples in real time according to certain embodiments in the provided disclosure. Here, FIG. 3 depicts a bottom view of the monitoring apparatus 300. As illustrated, the monitoring apparatus 300 may include a hollow body 305 and a weighted bottom 310. By way of example, the weighted bottom 310 may include an opening area exposing the sensor 315 to the solution when the monitoring apparatus 300 is placed in a body of water.

The sensor 315 may detect for specific qualities and characteristics important for maintaining ideal body of water conditions as determined by a user, such as oxygen concentration levels, pH, temperature and the like. However, it should be noted that the monitoring apparatus 300 is not limited to specific environmental conditions. In some embodiments, the sensor 315 may be a completely submersed in the body of water and obtain dissolved oxygen and temperature data of the water. In some instances, the sensor 315 may be an optical DO sensor. By way of example only, the sensor may include a miniDOT (e.g., PME sensor) using RS232 or USB to acquire the necessary data.

The sensor 315 may be configured to collect data in real time or within set time intervals, such as every minute, hour, or 12-hour intervals, by way of example only. The select time configuration may be determined and configured by the user. In some instances, the data collected from the sensor 315 may be transmitted to an offsite data storage server via the antenna 325. By way of example, the data collected from the monitoring apparatus 300 may be transmitted to the data storage server in real time or at select time intervals when the data is collected.

In other instances, the antenna 325 may send signals to other devices that regulate the specific environmental conditions of the aquaculture or contained body of water. For example, such devices may include an aerator, cooler, and/or heater. In some embodiments, when the sensor 315 detects oxygen measurements that are above or below a certain pre-determined range, a signal from the monitoring apparatus 300 may be transmitted to the aerator and cause the aerator to shut off. In other instances, when the sensor 315 detects temperature measurements that are above or below a certain pre-determined range, a signal from the monitoring apparatus 300 may be transmitted to the heater or cooler to raise or lower the temperature of the body of water.

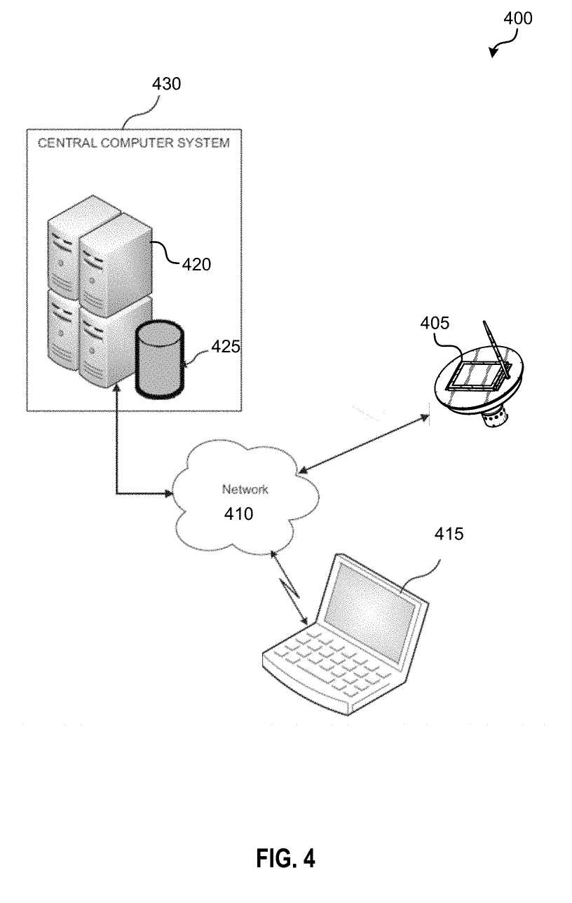

By way of another example, alerts may be generated and pushed to user operated devices, such as tablets, computers, laptops, smart phones, and the like. This may be performed by utilizing a cloud computing model 400, as illustrated in FIG. 4. By way of example, a user may access the web server software located on the central computer system 430 and create an alert associated with a user's pre-determined parameters. The user may connect to the network 410 via a laptop computer 415 or any other electronic device, such as a tablet, smart phone, desktop, etc. Furthermore, the monitoring apparatus 405 coupled to the network 410 via a wireless connection. The user may connect to the network 410 via a laptop computer 415 or any other electronic device, such as a tablet, smart phone, desktop, etc.

Additionally, the cloud computing model 400 may detect and analyze the received data and measurements from the monitoring apparatus 405. By way of example, when those data and measurements are below a user's set established pre-determined parameters, the cloud computing model 400 may send a signal to the monitoring apparatus 405, where the monitoring apparatus 405 may then send a signal and turn on, shut down, or adjust the necessary devices (e.g., aerator, cooler, heater, bubbler, etc.) controlling and maintaining the environment of the body of water.

The network 410 may connect the monitoring apparatus 405 and the laptop computer 415 to a server 420 and the database 425, which may store all of the data collected and transmitted from the sensor of the monitoring apparatus 405. The user may access the data collected from the monitoring apparatus 405 by accessing a web-based software via the network 410. The software may allow a user to view the collected data, set alarming options, and manage parameter set points. Thus, the web-based software may allow a user to create alerts so that when the data obtained from the monitoring apparatus 405 indicate sample measurements that are outside the determined parameter set points, an alert is sent to the user and the monitoring apparatus 405. Thus, this browser based format may allow a user to obtain an immediate overview of all of the body of water being monitored in real time.

In some embodiments, the server 420 may analyze the incoming data and measurements from the sensor of the computer control system 400 to detect for select data and measurements according to the user's specified alarm levels or rates of change. When such alarm levels or rates of change are detected from the gathered data, the server may provide a signal to notify the user. The user may be notified via SMS, email or other communication means. The end user may configure the notification settings based on how the user wishes to be notified of such alerts.

In some embodiments, the server may be configured to generate plots and graphs using the received data. For example, a temperature plot over some user selected time interval may be generated. This is can be configured so that such a temperature plot may be generated using yearly, monthly, weekly, daily, or even hourly time frames. Such a data plot may be used with other analytical data, such as pH levels, oxygen concentration levels, and the like. The data plot and/or graphs may be sent to the client in jpeg, pdf, or other image format that is generally compatible with general computing devices and/or mobile phones. Additionally, the client may request for specific image formats to be sent to his or her mobile device using the WiFi and Bluetooth connectivity arrangements with the sensor.

Referring back to FIG. 3, the monitoring apparatus 300 many include a wiper 320. The wiper 320 may be configured to rotate around the sensor 320 to automatically clean and swipe the surface of the sensor 320. By doing so, this may minimize and reduce algae growth on the sensor 315 and wipe away any dirt and other foreign particles located on the surface of the sensor 315. In some embodiments, the wiper 320 may be automated to rotate around the sensor 315 after a select pre-determined time period. For example, the wiper 320 may be configured to rotate around the sensor 315 every 3 hours, which may ensure that the sensor 315 is clean and able to take accurate measurements. However, it should be noted that the sensor 315 may be configured to clean the sensor 315 at any select time interval pre-determined by the user.

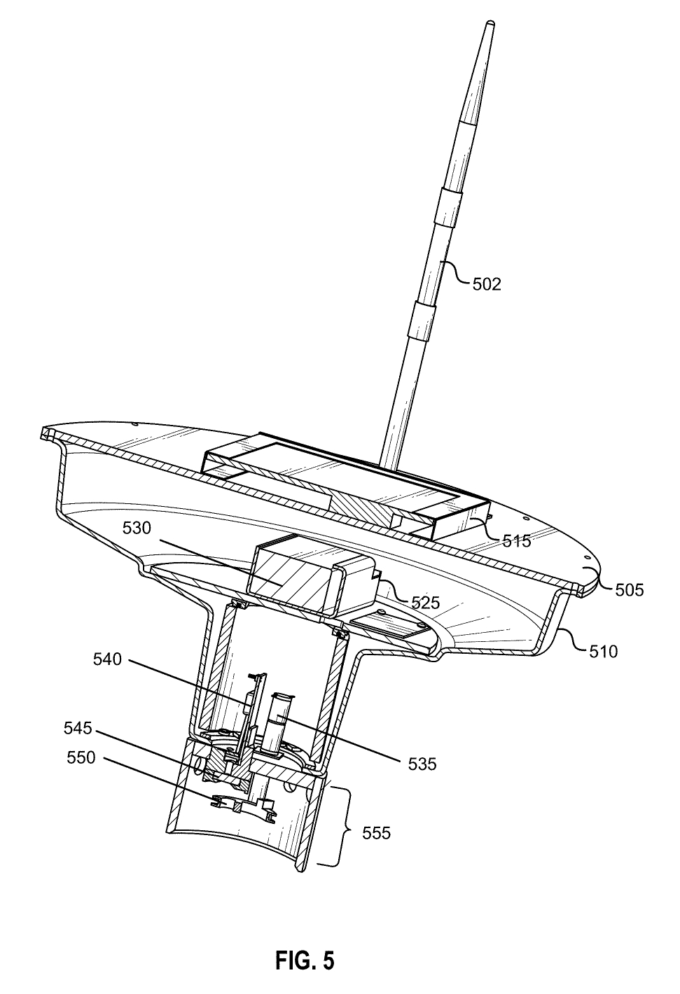

FIG. 5 is a cross-sectional view of a monitoring apparatus 500 that monitors solution samples in real time according to certain embodiments in the provided disclosure. As depicted, the monitoring apparatus 500 includes a hollow body 510 with an air-tight top cover 505. The top cover 505 may include a solar panel 515 to charge the internal battery 530 placed within the hollow body 510. The internal battery 530 may be securely held in place via a battery holder 525.

The fig. also discloses the wiper motor 535, which may be used to rotate the wiper 520 around the sensor 545. The wiper motor 535 may be connected to the internal battery 530, which may then provide the necessary power to run the wiper motor 435. Both the sensor electronics 540 and the wiper motor 535 may be placed above the weighted bottom 555, where the sensor electronics and wiper motor are placed in a sealed container. While the sensor electronics 540 and the wiper motor 535 are placed within a sealed container that is water proof, the sensor 545 and the wiper 550 may be positioned to be exposed to the water environment within the exposed area of the weighted bottom 555.

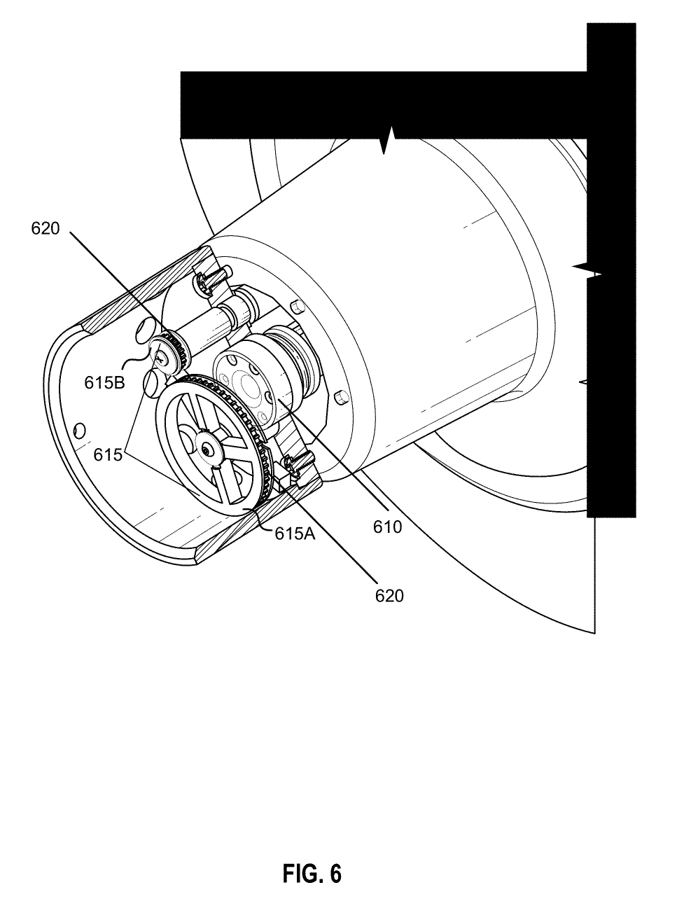

FIG. 6 is an illustration of a sensor 610 and a sensor wiper 615 according to certain embodiments of the provided disclosure. The sensor wiper 615 may be positioned beneath the sensor 610 so that the sensor wiper 615A may remove any debris accumulated beneath the sensor, such as algae or any other materials.

The sensor wiper 615 may be composed of a first wheel 615A and a second wheel 615B. The second wheel 615B may be connected to the motor (not shown here), as depicted in FIG. 5 within the tight seal portion within the monitoring apparatus. When the motor is turned on, the first wheel 615A may rotate, which may then cause the second wheel 615B directly beneath the sensor 610 to also rotate. Both the first wheel 615A and the second wheel 615B may include a set of raised edges 620, or teeth, that wrap around the entire circumference of the first wheel 615A and the second wheel 615B.

In some embodiments, the first wheel 615A of the sensor wiper 615 may include bristles located directly on top of first wheel 615A. Thus, when the second wheel 615A rotates, the bristles may not only remove any debris accumulated beneath the sensor 610, but may also come in contact with the sensor 610 and wipe away any debris, dirt, dust, or materials in contact with the surface of the sensor 610.

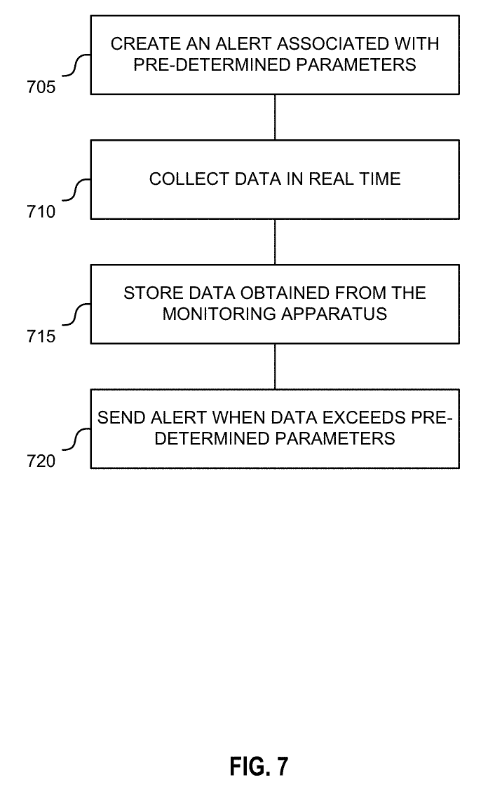

FIG. 7 is a flow chart of an exemplary process for monitoring a body of water according to certain embodiments of the provided disclosure. The exemplary process may include creating an alert associated with select pre-determined parameters at step 705. Thus, the pre-determined parameters may be selected to indicate specific water environment conditions the user considers to be ideal conditions. As a result, when the monitoring apparatus detects measurements that do not fall within the pre-determined parameters, an alert may be sent to the user and/or the monitoring apparatus. Thus, the alert may provide a quick way for users to be notified so that the water environment may quickly revert back to the ideal conditions.

Next, the process may include step 710, which is configuring the monitoring apparatus to collect data in real time. By way of example, as discussed above in detail, the monitoring apparatus may include a sensor or other equipment to measure temperature, oxygen content, pH and the like.

Next, the process may further include step 715, which may include storing the collected data obtain from the monitoring apparatus to a database in a central computer system. The data may be accessible to the user via an electronic device and connecting onto the appropriate network. By way of example, the data results may be viewed in a wide range of options, in which the user may choose the specific viewing options. For example, the data may be organized in the form of a graph, chart, table, list, summary format, and the like.

Next, the process may further include step 720, which may include sending an alert to a user or the monitoring apparatus when the data exceeds the pre-determined parameters. When such alarm levels or rates of change are detected from the gathered data, the server may provide a signal to notify the user. The user may be notified via SMS, email or other communication means. The end user may configure the notification settings based on how the user wishes to be notified of such alerts.

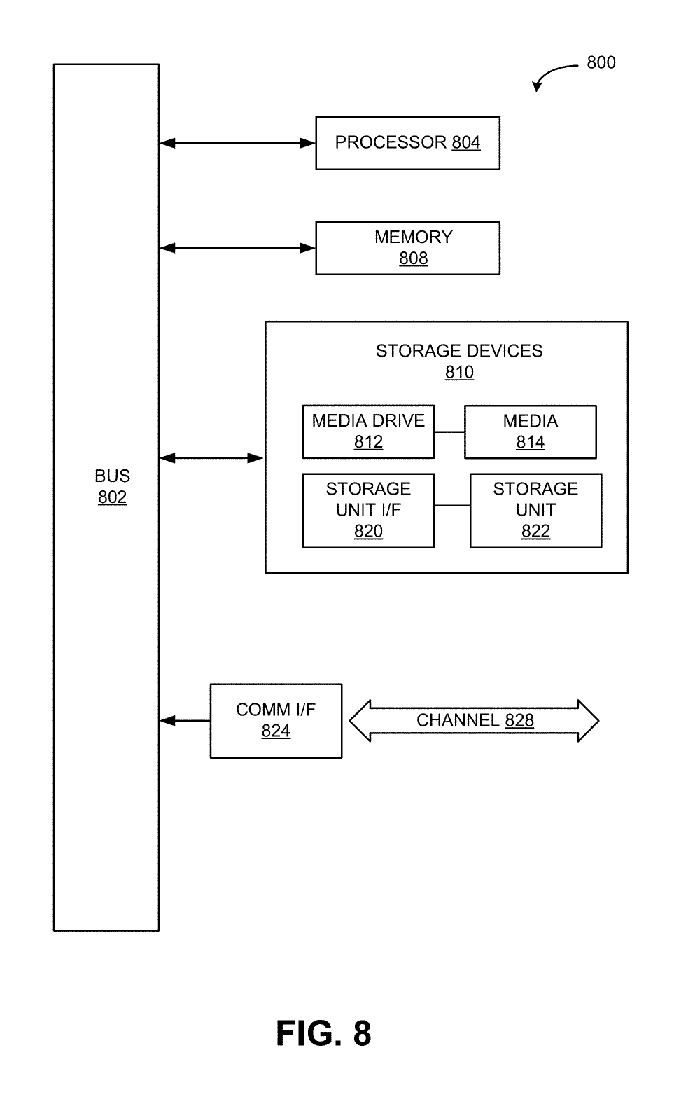

FIG. 8 illustrates example computing module 800, which may in some instances include a processor/controller resident on a computer system. Computing module 800 may be used to implement various features and/or functionality of embodiments of the systems and methods disclosed herein. With regard to the above-described embodiments, one of skill in the art will appreciate additional variations and details regarding the functionality of the embodiments, as set forth herein in the context of the apparatus and method described with reference to FIGS. 1 through 7. In this connection, it will also be appreciated by one of skill in the art that features and aspects of the various embodiments (e.g., systems) described herein may be implemented with respected to other embodiments (e.g., methods) described herein without departing from the spirit of the disclosure.

As used herein, the term module may describe a given unit of functionality that may be performed in accordance with one or more embodiments of the present application. As used herein, a module may be implemented utilizing any form of hardware, software, or a combination thereof. For example, one or more processors, controllers, ASICs, PLAs, PALs, CPLDs, FPGAs, logical components, software routines or other mechanisms may be implemented to make up a module. In implementation, the various modules described herein may be implemented as discrete modules or the functions and features described may be shared in part or in total among one or more modules. In other words, as would be apparent to one of ordinary skill in the art after reading this description, the various features and functionality described herein may be implemented in any given application and may be implemented in one or more separate or shared modules in various combinations and permutations. Even though various features or elements of functionality may be individually described or claimed as separate modules, one of ordinary skill in the art will understand that these features and functionality may be shared among one or more common software and hardware elements, and such description shall not require or imply that separate hardware or software components are used to implement such features or functionality.

Where components or modules of the application are implemented in whole or in part using software, in one embodiment, these software elements may be implemented to operate with a computing or processing module capable of carrying out the functionality described with respect thereto. One such example computing module is shown in FIG. 8. Various embodiments are described in terms of example computing module 800. After reading this description, it will become apparent to a person skilled in the relevant art how to implement the application using other computing modules or architectures.

Referring to FIG. 8, computing module 800 may represent, for example, computing or processing capabilities found within mainframes, supercomputers, workstations or servers; desktop, laptop, notebook, or tablet computers; hand-held computing devices (tablets, PDA's, smartphones, cell phones, palmtops, etc.); or the like, depending on the application and/or environment for which computing module 800 is specifically purposed.

Computing module 800 may include, for example, one or more processors, controllers, control modules, or other processing devices, such as a processor 804. Processor 804 may be implemented using a special-purpose processing engine such as, for example, a microprocessor, controller, or other control logic. In the illustrated example, processor 804 is connected to bus 802, although any communication medium may be used to facilitate interaction with other components of computing module 800 or to communicate externally.

Computing module 800 may also include one or more memory modules, simply referred to herein as main memory 808. For example, random access memory (RAM) or other dynamic memory may be used for storing information and instructions to be executed by processor 804. Main memory 808 may also be used for storing temporary variables or other intermediate information during execution of instructions to be executed by processor 804. Computing module 800 may likewise include a read only memory (ROM) or other static storage device coupled to bus 802 for storing static information and instructions for processor 804.

Computing module 800 may also include one or more various forms of information storage devices 810, which may include, for example, media drive 812 and storage unit interface 820. Media drive 812 may include a drive or other mechanism to support fixed or removable storage media 814. For example, a hard disk drive, a floppy disk drive, a magnetic tape drive, an optical disk drive, a CD or DVD drive (R or RW), or other removable or fixed media drive may be provided. Accordingly, removable storage media 814 may include, for example, a hard disk, a floppy disk, magnetic tape, cartridge, optical disk, a CD or DVD, or other fixed or removable medium that is read by, written to or accessed by media drive 812. As these examples illustrate, removable storage media 814 may include a computer usable storage medium having stored therein computer software or data.

In alternative embodiments, information storage devices 810 may include other similar instrumentalities for allowing computer programs or other instructions or data to be loaded into computing module 800. Such instrumentalities may include, for example, fixed or removable storage unit 822 and storage unit interface 820. Examples of such removable storage units 822 and storage unit interfaces 820 may include a program cartridge and cartridge interface, a removable memory (for example, a flash memory or other removable memory module) and memory slot, a PCMCIA slot and card, and other fixed or removable storage units 822 and storage unit interfaces 820 that allow software and data to be transferred from removable storage unit 822 to computing module 800.

Computing module 800 may also include a communications interface 824. Communications interface 824 may be used to allow software and data to be transferred between computing module 800 and external devices. Examples of communications interface 824 include a modem or softmodem, a network interface (such as an Ethernet, network interface card, WiMedia, IEEE 802.XX or other interface), a communications port (such as for example, a USB port, IR port, RS232 port Bluetooth® interface, or other port), or other communications interface. Software and data transferred via communications interface 824 may typically be carried on signals, which may be electronic, electromagnetic (which includes optical) or other signals capable of being exchanged by a given communications interface 824. These signals may be provided to communications interface 824 via channel 828. Channel 328 may carry signals and may be implemented using a wired or wireless communication medium. Some non-limiting examples of channel 828 include a phone line, a cellular link, an RF link, an optical link, a network interface, a local or wide area network, and other wired or wireless communications channels.

In this document, the terms “computer program medium” and “computer usable medium” are used to generally refer to transitory or non-transitory media such as, for example, main memory 808, storage unit interface 820, removable storage media 814, and channel 828. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions may enable the computing module 800 or a processor to perform features or functions of the present application as discussed herein.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

What is claimed is:

1. A monitoring apparatus comprising:
a hollow body;
a top cover to seal the hollow body to provide a waterproof monitoring apparatus;
a weighted bottom portion beneath the hollow body; and
a sensor exposed to an environment to monitor and acquire data from a solution sample.

2. The monitoring apparatus of claim 1, wherein the top cover comprises a solar panel to charge an internal battery placed in the hollow body.

3. The monitoring apparatus of claim 1, further comprising an antenna to transmit data collected from the sensor to a database.

4. The monitoring apparatus of claim 1, further comprising a status indicator on the top cover to provide visual indication of the condition of the environment based on data acquired from the sensor.

5. The monitoring apparatus of claim 4, wherein the sensor detects at least any one of oxygen concentration, pH and temperature of the environment.

6. The monitoring apparatus of claim 5, wherein the sensor collects data at select pre-determined time intervals.

7. The monitoring apparatus of claim 5, wherein the sensor collects data in real-time.

8. The monitoring apparatus of claim 1, wherein the monitoring apparatus further comprises a sensor cleaner located beneath the sensor to wipe a surface of the sensor.

9. The monitoring apparatus of claim 7, wherein the sensor cleaner wipes a surface of the sensor at select pre-determined time intervals.

10. A system for monitoring solution samples, comprising:
a monitoring apparatus comprising:
a hollow body;
a top cover to seal the hollow body to provide a waterproof monitoring apparatus;
a weighted bottom portion beneath the hollow body; and
a sensor exposed to an environment to monitor and acquire data from a solution sample;
a server in wireless communication with the monitoring apparatus to store data transmitted from the sensor;
wherein the server sends an alert to a user when the server detects data collected from the monitoring apparatus is not within a range of pre-determined parameters;
an electronic device connected to a network to access data stored in the server; and
one or more device regulating the environment in communication with the monitoring apparatus or the server, such that the device is controlled by the server in response to the data collected from the monitoring apparatus.

11. The system of claim 10, wherein the monitoring apparatus further comprises a status indicator on the top cover to provide visual indication of the condition of the environment based on data acquired from the sensor.

12. The system of claim 10, wherein the monitoring apparatus further comprises a solar panel to charge an internal battery placed in the hollow body.

13. The system of claim 10, wherein the sensor detects at least any one of oxygen concentration, pH and temperature of the environment.

14. The system of claim 13, wherein the sensor collects data at select pre-determined time intervals.

15. The system of claim 10, wherein the sensor collects data in real-time.

16. The system of claim 10, wherein the monitoring apparatus further comprises a sensor cleaner located beneath the sensor to wipe a surface of the sensor at predetermined time intervals.

17. A method of obtaining solution sample measurements comprising:
creating an alert associated with pre-determined parameters for maintaining target environment conditions;
collecting data in real time with a monitoring apparatus placed in an environment, wherein the monitoring apparatus comprises:
a hollow body;
a top cover to seal the hollow body to provide a waterproof monitoring apparatus;
a weighted bottom portion beneath the hollow body; and
a sensor exposed to an environment to monitor and acquire data from a solution sample;
storing data obtained from the monitoring apparatus; and
sending alert to a user when obtained data from the monitoring apparatus exceeds the pre-determined parameters.

18. The method of claim 17, wherein the sensor detects at least any one of oxygen concentration, pH and temperature of the environment.

19. The method of claim 17, wherein the sensor collects data at select pre-determined time intervals.

20. The method of claim 17, further comprising rotating a sensor wiper around a surface of a sensor at select pre-determined time intervals.
